(19) United States
(12) Patent Application Publication
(10) Pub. No.: US 2026/0199601 A1
(43) Pub. Date: Jul. 16, 2026

(54) WEARABLE DUAL CLOSED-LOOP INSULIN DELIVERY SYSTEMS FOR PRECISION DIABETES MANAGEMENT

(71) Applicant: The University of Hong Kong, Hong Kong (HK)

(72) Inventors: Shiming ZHANG, Hong Kong (HK); Xuecheng HE, Hong Kong (HK); Wei HUANG, Hong Kong (HK)

(21) Appl. No.: 19/448,112

(22) Filed: Jan. 14, 2026

Related U.S. Application Data
(60) Provisional application No. 63/746,228, filed on Jan. 16, 2025.

(51) Int. Cl. A61M 5/172; A61K 47/64

(52) U.S. Cl. CPC A61M 5/1723; A61K 47/6455; A61M 2005/1726; A61M 2202/04; A61M 2205/3303; A61M 2230/201

(57) ABSTRACT

A wearable dual closed-loop insulin delivery system is provided that integrates both a chemical closed-loop and an electronic closed-loop for precise glycemic control. The system includes a continuous glucose monitor (CGM) configured to obtain real-time blood glucose concentrations of a subject, and a glucose-responsive insulin (GRI) delivery device containing a GRI formulation that autonomously modulates its insulin-release rate in response to the subject's glucose levels, thereby forming a chemical closed-loop. The system further includes a controller having one or more computer processors executing a machine-learning adaptive glucose forecasting model with an Encoder-Decoder architecture. The model receives real-time glucose measurements from the CGM, predicts glycemia dynamics for at least a time period (i.e., 30 minute) future interval, and generates dosing-control signals to adjust both insulin-dosing amount and timing, forming an electronic closed-loop. The controller is operatively connected to the CGM and the GRI delivery device, enabling predictive, automated insulin regulation.

### CROSS-REFERENCE TO RELATED APPLICATIONS

The present application claims priority from U.S. Provisional Utility Patent application No. 63/746,228 filed Jan. 16, 2025; the disclosure of which is incorporated herein by reference in its entirety.

### FIELD OF THE INVENTION

The present invention generally relates to the field of diabetes management technologies. More specifically the present invention relates to wearable dual closed-loop insulin delivery systems integrating chemical and electronic closed-loop for regulation of blood glucose.

### BACKGROUND OF THE INVENTION

Diabetes is a chronic metabolic disease characterized by impaired insulin secretion and persistent hyperglycemia. The disease is projected to affect approximately 643 million individuals worldwide by 2030. Conventional diabetes management relies on separate glucose monitoring and insulin administration. However, this approach often results in suboptimal glycemic control and may lead to severe complications, including hypoglycemia, seizures, loss of consciousness, or even death, particularly when excessive insulin is administered.

Continuous glucose monitoring (CGM)-controlled closed-loop insulin delivery systems have recently emerged as a more effective approach for maintaining blood glucose levels within a desired range. In such systems, a CGM sensor continuously measures glucose concentrations, and a control algorithm adjusts insulin delivery based on real-time glucose data and carbohydrate intake. Despite these advances, electronic closed-loop systems continue to face safety concerns. In particular, inaccurate or false glucose readings can result in excessive insulin delivery, increasing the risk of dangerous hypoglycemic events.

To address these limitations, glucose-responsive insulin (GRI) formulations have been developed. GRIs typically rely on one of three glucose-responsive mechanisms—glucose-binding proteins, glucose oxidase, or phenylboronic acid—to regulate insulin release in response to ambient glucose levels, thereby mimicking the physiological behavior of pancreatic β cells. Existing GRIs, however, are commonly administered through disposable patches, oral dosage forms, or injections. Due to their limited self-regulation capability, these administration modalities can still give rise to episodes of hyperglycemia or hypoglycemia.

Integrating electronic closed-loop control with chemical closed-loop GRI systems has the potential to provide more precise control of insulin dosing and timing, thereby improving therapeutic outcomes. However, the development of a reliable and intelligent algorithm capable of coordinating these two systems remains a significant technical challenge. Accordingly, there remains a need for improved systems and methods for achieving safe, accurate, and responsive glucose-regulated insulin delivery. The present invention is directed toward satisfying this need.

### SUMMARY OF THE INVENTION

It is an objective of the present invention to provide systems and methods to solve the aforementioned technical problems.

In accordance with a first aspect of the present invention, a wearable dual closed-loop insulin delivery system integrating a chemical closed-loop and an electronic closed-loop is provided. Specifically, the wearable dual closed-loop insulin delivery system includes: a CGM configured to obtain a series of real-time blood glucose concentrations of a subject; a GRI delivery device configured to administer insulin to the subject at a release rate modulated by the real-time blood glucose concentrations, including a GRI formulation that autonomously modulates its insulin-release rate in response to the subject's real-time glucose concentration, thereby forming a chemical closed-loop; and a controller including one or more computer processors and a machine-learning adaptive glucose forecasting model implemented thereon.

The model comprising an Encoder-Decoder architecture configured to: receive the series of real-time glucose concentrations from the CGM; generate a predicted glycemia dynamics of the subject for at least a subsequent time period (i.e., 30 minute) prediction horizon; and generate dosing-control signals based on the predicted glycemia dynamics to adjust an insulin-dosing amount and an insulin-dosing timing of the GRI delivery device, thereby forming an electronic closed-loop. The controller is operatively connected to the CGM to receive the real-time glucose concentrations and operatively connected to the GRI delivery device to transmit the dosing-control signals.

In accordance with one embodiment, the wearable system is compatible with a smart device including a smartphone, a smartwatch, and smart glasses.

In accordance with another embodiment, the machine-learning adaptive glucose forecasting model is pre-trained using a dataset including continuous glucose monitoring data and insulin-administration records.

In accordance with yet another embodiment, the GRI delivery device reduces the occurrence of hypoglycemia by automatically decreasing insulin release in response to a glucose level declining event in the series of real-time glucose concentrations.

In accordance with yet another embodiment, the Encoder includes two self-attention layers and the Decoder includes a masked self-attention layer followed by a self-attention layer.

In accordance with yet another embodiment, the continuous glucose monitoring data includes at least 100 minutes of historical CGM glucose measurements together with corresponding insulin-administration records provided as input to pre-train the machine-learning adaptive glucose forecasting model.

In accordance with yet another embodiment, the machine-learning adaptive glucose forecasting model communicates the predicted glycemia dynamics to the GRI delivery device via a wireless communication interface.

In accordance with yet another embodiment, the controller further includes a proportional-integral-derivative (PID) controller executed by the computer processors and configured to receive the predicted glycemia dynamics and generate the dosing-control signals.

In accordance with yet another embodiment, the CGM includes an organic electrochemical transistor (OECT) integrated with a microneedle array for interstitial fluid extraction.

In accordance with yet another embodiment, the CGM detects glucose concentrations in a range from 0.2 mM to 40 mM.

In accordance with yet another embodiment, detection of a predicted hyperglycemia event within the predicted glycemia dynamics triggers a pre-emptive insulin-delivery action by the GRI delivery device to prevent or mitigate hyperglycemia.

In accordance with yet another embodiment, the GRI formulation includes gluconic-acid-modified insulin complexed with poly-L-lysine functionalized with phenylboronic acid groups.

In accordance with a second aspect of the present invention, a method for regulating insulin delivery in a subject using the aforementioned system is provided. Particularly, the method includes the following steps: obtaining a series of real-time glucose measurements from the CGM; predicting glucose levels for at least a subsequent time period (i.e., 30 minute) using the machine-learning adaptive glucose forecasting model; and modulating glucose-responsive insulin release from the GRI delivery device based on the predicted glucose levels so as to maintain euglycemia in the subject.

In accordance with one embodiment, detection of a predicted hyperglycemia event within the predicted glucose levels causes the controller to initiate a pre-emptive GRI-dosing command to the glucose-responsive insulin delivery device to prevent or reduce hyperglycemic excursions.

In accordance with another embodiment, detection of a predicted hypoglycemia event within the predicted glucose levels causes the controller to suppress, reduce, or temporarily suspend insulin release from the glucose-responsive insulin delivery device to avoid hypoglycemic undershoot.

### BRIEF DESCRIPTION OF THE DRAWINGS

Embodiments of the invention are described in more details hereinafter with reference to the drawings, in which:

FIGS. 1A-1E depict the overall design of DuoLoop, a wearable dual closed-loop insulin delivery system according to one embodiment of the present invention, in which: FIG. 1A shows the primary components of the DuoLoop system, including the CGM, control algorithm, and insulin pump operating within the human body; FIG. 1B depicts the functional relationships and communication pathways among these components; FIG. 1C illustrates the workflow and operating mechanism of the DuoLoop system; FIG. 1D provides a comparative schematic of glucose dynamics demonstrating differences between a conventional single closed-loop device and the DuoLoop system; and FIG. 1E depicts a wearable dual closed-loop insulin delivery system in according to one embodiment of the present invention;

FIGS. 2A-2E depict the synthesis and characterization of GRI, in which: FIG. 2A is a schematic representation of the glucose-responsive mechanism of the polymer-insulin complex; FIG. 2B depicts that BG-triggered insulin release following intraperitoneal glucose injection in diabetic rats with solid lines indicating blood glucose (mM) and dash lines indicating serum glucose (mU L⁻¹); FIG. 2C and FIG. 2D depicts the ISF glucose levels in diabetic rats after a single injection (25 U/kg) of HRI (FIG. 2C) and GRI (FIG. 2D); and FIG. 2E shows the corresponding normoglycemia duration of FIGS. 2C-2D;

FIGS. 3A-3C depict the algorithm development for DuoLoop system, in which: FIG. 3A shows the quantized edge-AI implementation of the dual closed-loop system using a proportional-integral-derivative (PID) learning framework; FIG. 3B depicts the validation of the glucose prediction algorithm on a simulated virtual patient; and FIG. 3C displays the validation of the insulin injection algorithm on a simulated virtual patient;

FIGS. 4A-4D depict the validation of the edge-AI embedded dual closed-loop system, in which: FIG. 4A shows the experimental validation of the algorithm by comparing the predicted glycemia dynamics with the actual outcomes in diabetic rats: FIG. 4B, FIG. 4C and FIG. 4D depict the experimental validation of the algorithm by comparing glycemia dynamics under two conditions: delivery at the algorithm-recommended dose (FIG. 4B and FIG. 4C) and delivery at a deliberately altered time and dose (FIG. 4D);

FIGS. 5A-5J depict the results of in vivo animal experiments, in which: FIG. 5A depicts the schematic (left) and actual images (right) of the validation of DuoLoop in a type 1 diabetic rat model; FIG. 5B depicts the edge-AI-powered dual closed-loop system; FIG. 5C and FIG. 5D depict the pure chemical closed-loop treatment of diabetic rats through regular GRI injections (5 U/kg) administered every 7 ours (FIG. 5C) and 8 hours (FIG. 5D); FIG. 5E and FIG. 5F show the comparisons of the combinational ISF glucose levels in diabetic rats treated with SinLoop (FIG. 5E) and DuoLoop (FIG. 5F); FIG. 5G, FIG. 5H, FIG. 5I, and FIG. 5J show the analysis of coefficient of variation (FIG. 5G), normoglycemia percentage (FIG. 5H), hyperglycemia percentage (FIG. 5I), and hypoglycemia percentage (FIG. 5J) in diabetic rats treated with SinLoop (labeled “Single”) and DuoLoop (labeled “Dual”);

FIGS. 6A-6D depict the characterization of the microneedle, in which: FIGS. 6A and 6B are SEM images showing an array of multiple microneedles (FIG. 6A) and a single microneedle (FIG. 6B); FIG. 6C shows the finite element analysis of microneedle insertion into the skin; and FIG. 6D depicts the force-displacement curve of the microneedle;

FIGS. 7A-7B depict the structural layout of the OECT backup CGM, in which: FIG. 7A shows an exploded view of the subcomponents of the OECT CGM, including a hollow microneedle patch, an OECT, a personalized electronic reader for electrochemical transistors (PERfECT), and a foldable flexible printed circuit board (fPCB) connector, serving as the power line connecting the OECT sensor to the PERfECT system; and FIG. 7B depicts the circuit diagram and key components of the PERfECT system;

FIGS. 8A-8C depict the characterization of the OECT, in which: FIG. 8A shows the transfer curves with Vg scanned between −0.2 and 0.8 V while Vd remained fixed for each curve; FIG. 8B shows the corresponding transconductance; and FIG. 8C depicts the output characteristics with VdV_dVd scanned from 0 to 1 V and Vg fixed for each curve;

FIGS. 9A-9C depict the glucose detection using the OECT backup CGM, in which: FIG. 9A shows the schematic illustrating glucose diffusion mechanics between the ISF and the OECT via the hollow microneedle: FIG. 9B is a schematic of the OECT-based glucose sensing mechanism, where the GOx-modified gate electrode catalyzes glucose decomposition, facilitated by a ferrocene mediator, so as to promote electron transport at the gate electrode, resulting in a glucose-dependent variation in current (ΔIds) between the source and drain electrodes; and FIG. 9C shows the real-time current response (ΔIds) of the OECT-based glucose sensor to glucose concentrations ranging from 0 to 40 mM, with the inset highlighting the detection of low glucose concentrations (0-1 mM);

FIGS. 10A-10B depict the construction of type 1 diabetic rats, comparing the glucose dynamics in between healthy rats (FIG. 10A) and diabetic rats (FIG. 10B) without insulin treatment;

FIG. 11 depicts the one-to-one correlation between blood glucose and interstitial fluid glucose levels, where the dashed line represents the line of identity;

FIG. 12 depicts the synthesis route of PEG-PLL-FPBA;

FIG. 13 depicts the ¹H NMR spectrum of FPBA-NHS in DMSO-d6;

FIG. 14 depicts the chemical structure of the polymer and its corresponding reaction mechanism with glucose;

FIG. 15 depicts the TEM image of polymer-insulin complex nanomicelles;

FIG. 16 depicts the in vivo glucose tolerance test in diabetic rats 2 hours after the administration of RHI or GRI injections;

FIG. 17 depicts the glycemic profiles of diabetic rats treated with varying doses of HRI and GRI;

FIGS. 18A-18B depict the construction of the insulin pump, in which: FIG. 18A shows the optical image of the customized insulin pump and FIG. 18B is a functional block diagram highlighting the major electronic components;

FIGS. 19A-19C depict the characterizations of the customized insulin pump, in which: FIG. 19A shows the correlation between pump power and solution delivery speed; FIG. 19B depicts the consistency between the actual delivered volume and the preset volume of the customized insulin pump; and FIG. 19C depicts the user interface of the insulin pump control system;

FIGS. 20A-20B depict the trial profiles of the glucose prediction Transformer model (FIG. 20A) and the AI-powered insulin delivery system in silicon-based patient simulations (FIG. 20B);

FIGS. 21A-21C depict the validation of the insulin injection algorithm on simulated virtual patients in multiple times; and

FIG. 22 displays the representative images of Hematoxylin & Eosin (H&E, top) and Masson's trichrome (MTC, bottom)-stained sections of the full-thickness insulin delivery site over 7 days of treatment.

### DETAILED DESCRIPTION

In the following description, wearable dual closed-loop insulin delivery systems and/or methods for regulating insulin delivery and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

As used herein, the term “continuous glucose monitoring data” refers to a time-resolved sequence of glucose measurements collected at regular intervals by a CGM. The data represent dynamic changes in a subject's interstitial fluid glucose or blood glucose levels over a monitoring period. Such data typically include:
(1) numerical glucose values measured at fixed sampling intervals (e.g., every 1-5 minutes);
(2) corresponding timestamps;
(3) optionally associated sensor metadata (e.g., signal quality, calibration parameters); and
(4) trends or rate-of-change information derived from raw CGM readings.

As used herein, the term “continuous glucose monitor (CGM)” refers to a wearable or implantable sensor system capable of continuously or semi-continuously measuring glucose concentrations in a subject's interstitial fluid or blood at regular time intervals. A CGM typically includes: a glucose-sensing element (e.g., enzymatic, electrochemical, or transistor-based sensor), a data acquisition unit configured to generate time-resolved glucose readings, a processor for converting sensor signals into glucose concentrations, and a transmitter for communicating glucose data to an external device, such as a controller, insulin pump, or smart device. The CGM provides real-time glucose measurements, glucose trends, and/or rate-of-change information to enable continuous glycemic monitoring and electronic closed-loop insulin regulation.

As used herein, the term “glucose-responsive insulin (GRI) delivery device” refers to a device configured to administer a glucose-responsive insulin formulation to a subject, wherein the rate or amount of insulin released is modulated by the subject's real-time glucose concentration. The device typically includes: a reservoir containing a GRI formulation whose insulin-release profile changes in response to elevated or falling glucose levels; a dispensing or injection mechanism configured to deliver the GRI formulation into the subject; and control circuitry or communication components enabling adjustment of delivery parameters based on glucose measurements, predicted glucose trends, or both. The GRI delivery device operates as part of a chemical closed-loop system, providing self-regulated insulin release driven by biochemical interactions between the GRI formulation and the subject's glucose levels, thereby reducing the risk of hypoglycemia and hyperglycemia.

As used herein, the term “chemical and electronic closed-loop control” refers to a dual-regulation system in which insulin delivery is simultaneously governed by the aforementioned chemical closed-loop and electronic closed-loop. Together, the chemical loop provides biochemical self-regulation of insulin release, while the electronic loop provides algorithm-controlled predictive regulation, thereby forming a synergistic dual-loop feedback system designed to maintain stable blood-glucose levels and reduce risks of hypoglycemia and hyperglycemia.

As used herein, the term “hyperglycemia event” refers to an occurrence during which a subject's blood glucose level rises above a predefined hyperglycemic threshold for a measurable or predicted duration of time. A hyperglycemia event may indicate inadequate insulin delivery, delayed glucose-responsive insulin activation, or an impending glucose excursion, and is used in closed-loop systems to trigger corrective insulin-dosing actions or predictive adjustments.

As used herein, the term “hypoglycemia event” refers to an occurrence in which a subject's blood glucose level falls below a predefined hypoglycemic threshold for a measurable or predicted period of time. A hypoglycemia event may result from excessive insulin delivery, delayed glucose intake, increased insulin sensitivity, or metabolic variability, and is used in closed-loop control systems to trigger suppression or suspension of insulin delivery and initiate corrective safety actions.

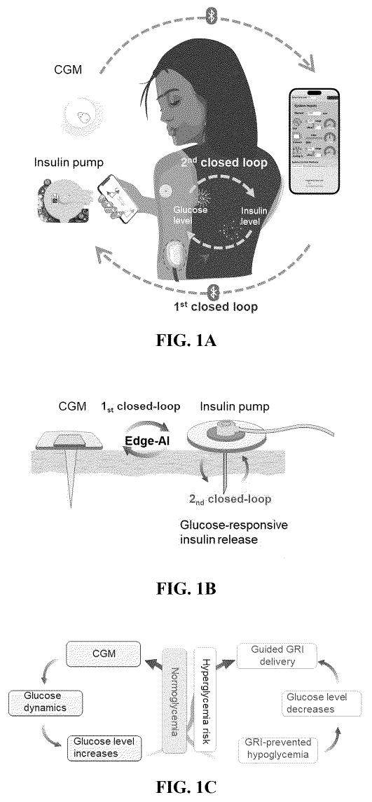

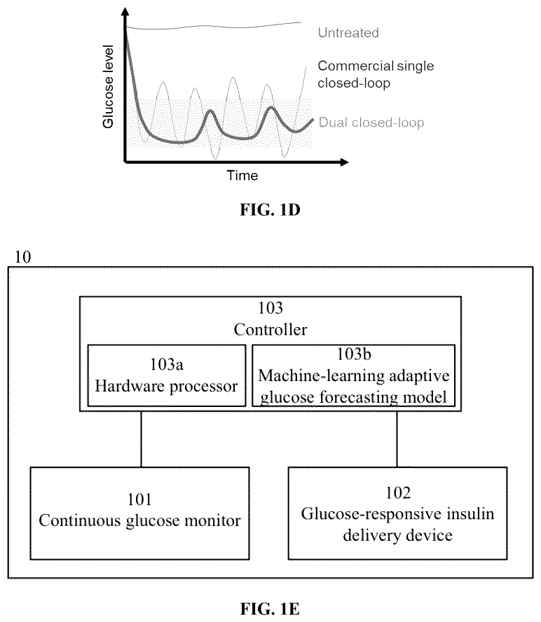

In accordance with a first aspect of the present invention, a wearable dual closed-loop insulin delivery system 10 that integrates both a chemical closed-loop and an electronic closed-loop to achieve precise, adaptive, and safe regulation of insulin in a subject is provided (FIG. 1E). The system 10 includes a CGM 101 configured to obtain a series of real-time blood glucose concentrations from the subject. These measurements may be derived from interstitial fluid or blood and are continuously streamed to a controller. The system 10 further includes a GRI delivery device 102, which administers insulin to the subject at a release rate modulated directly by the subject's real-time glucose concentration. In certain embodiments, the GRI delivery device 102 incorporates a GRI formulation that autonomously adjusts its insulin-release rate in response to dynamic fluctuations in glucose levels, thereby forming an intrinsic chemical closed-loop.

Coordinated with this chemical loop is an electronic closed-loop implemented by a controller 103 including one or more computer processors 103a and a machine-learning adaptive glucose forecasting model 103b executed thereon. The machine-learning model 103b utilizes an Encoder-Decoder architecture configured to receive the real-time glucose concentrations from the CGM 101, process the time-series inputs, and generate a predicted glycemia dynamics profile of the subject for at least a subsequent time period (i.e., 30 minute) prediction horizon. Based on the predicted glucose trajectory, the model 103b produces dosing-control signals that adjust the insulin-dosing amount and dosing timing of the GRI delivery device 102. Through this dual control mechanism, the controller 103 maintains communication with the CGM 101 to receive glucose data and transmits control instructions to the GRI delivery device 102 to regulate insulin delivery before glycemic excursions occur.

In certain embodiments, the wearable system is compatible with smart devices including smartphones, smartwatches, and smart glasses, enabling mobile visualization, user notifications, remote monitoring, or external computational support. The machine-learning adaptive glucose forecasting model may be pre-trained using datasets that include continuous glucose monitoring data and corresponding insulin-administration records, thereby enabling personalized or population-based predictive capabilities. The CGM data used for such pre-training may include at least 100 minutes of historical glucose measurements together with paired insulin-administration information to provide sufficient temporal context.

In some embodiments, the GRI delivery device is configured to reduce the occurrence of hypoglycemia by automatically decreasing insulin release in response to a glucose-level declining event detected in the real-time CGM stream. The Encoder module of the forecasting model may include two self-attention layers to capture long-range temporal dependencies, while the Decoder includes a masked self-attention layer followed by an additional self-attention layer to generate accurate next-interval predictions.

Communication between the machine-learning adaptive glucose forecasting model and the GRI delivery device may occur via a wired or wireless communication interface, allowing wearable components to operate flexibly without physical tethering. The controller may also include a proportional-integral-derivative (PID) controller executed by the computer processors. The PID controller receives the predicted glycemia dynamics and transforms the prediction into dosing-control signals that modulate both the quantity and timing of insulin delivery.

In certain embodiments, the CGM includes an organic electrochemical transistor (OECT) integrated with a microneedle array designed for interstitial fluid extraction. Such CGM designs may enable detection of glucose concentrations across a broad physiological range, for example from 0.2 mM to 40 mM, accommodating both hypoglycemic and hyperglycemic states. When the predicted glycemia dynamics include a predicted hyperglycemia event, the controller may automatically initiate a pre-emptive insulin-delivery action through the GRI device to prevent or mitigate hyperglycemia before it becomes clinically significant.

The GRI formulation used in the GRI delivery device may include a gluconic-acid-modified insulin complexed with poly-L-lysine functionalized with phenylboronic acid groups. The formulation is capable of dynamically altering its insulin release rate based on glucose binding interactions, thereby providing a chemical closed-loop effect that complements the predictive and command-based regulation of the electronic closed-loop.

Taken together, the integrated wearable system combines chemical self-regulation with machine-learning-driven predictive control to deliver a robust and adaptive insulin-management platform capable of reducing glycemic fluctuations, preventing hyperglycemia and hypoglycemia events, and improving overall glucose control in diabetic subjects.

In accordance with a second aspect of the present invention, a method for regulating insulin delivery in a subject utilizing the aforementioned wearable dual closed-loop insulin delivery system. In this method, a series of real-time glucose measurements is obtained from the CGM, which continuously samples the subject's interstitial fluid or blood glucose levels to generate a time-resolved glucose profile. These real-time measurements are processed using the machine-learning adaptive glucose forecasting model to generate a prediction of the subject's glucose levels for at least a subsequent time period (i.e., 30 minute). Based on this predicted glycemic trajectory, the glucose-responsive insulin release from the GRI delivery device is modulated so as to maintain euglycemia in the subject. In this way, the insulin delivery method leverages both real-time physiological sensing and anticipatory prediction to adjust insulin administration before large glucose excursions occur.

In some embodiments of the method, when the predicted glucose values indicate the onset of a hyperglycemia event, the controller detects the predicted hyperglycemic excursion and proactively initiates a pre-emptive GRI-dosing command to the glucose-responsive insulin delivery device. This anticipatory activation is configured to prevent or reduce hyperglycemia by delivering insulin prior to glucose levels rising beyond the euglycemic range. Conversely, in embodiments where the predicted glucose trajectory indicates a forthcoming hypoglycemia event, the controller detects the predicted hypoglycemic trend and responds by suppressing, reducing, or temporarily suspending insulin release from the glucose-responsive insulin delivery device. This preventative modulation avoids hypoglycemic undershoot and maintains glucose levels within a safe physiological range. Through this integrated, predictive, and adaptive regulation, the method enables precise glycemic control with reduced risk of both hyperglycemia and hypoglycemia.

### EXAMPLES

### Example 1. Wearable System Integrating Chemical and Electronic Closed-Loop Control for Insulin Delivery

A wearable system integrating chemical and electronic closed-loop control for insulin delivery, referred to as the DuoLoop system, is developed according to one embodiment of the present invention. The DuoLoop system is designed to address the safety limitations of traditional single closed-loop (SinLoop) systems (FIG. 1A). The DuoLoop system includes three primary components: a CGM for real-time glucose sensing; an insulin pump for delivering GRI (the GRI delivery device); and a pre-trained machine-learning-based adaptive glucose forecasting model that operates an adaptive control algorithm. The forecasting model is trained on 100 specialized experimental GRI datasets and is capable of guiding insulin delivery by forecasting impending glycemic dynamics based on real-time CGM readings.

Unlike the SinLoop system, the DuoLoop incorporates dual closed-loop regulation, substantially reducing safety risks. First, the algorithm-enabled edge AI predicts glucose trends some time period (i.e., 30 minutes) in advance, enabling proactive adjustment of GRI administration (first loop). Second, the actual release rate of the injected GRI is further modulated by the patient's real-time blood glucose levels (second loop), ensuring autonomous biochemical self-regulation. (FIGS. 1B-1C)

As shown in FIG. 1D, animal studies involving six experimental groups and 1,620 CGM datasets validate the superior precision and safety of the DuoLoop relative to a traditional SinLoop system. The DuoLoop achieves markedly improved glycemic outcomes, including significantly longer durations within the normoglycemic range (98.82% vs. 92.10%), reduced incidence of hyperglycemia (0.65% vs. 3.89%), minimized hypoglycemia (0.52% vs. 4.01%), and substantially lower glucose variability (coefficient of variation: 25.14 vs. 41.22).

### Example 2. Calibration of Wearable CGM

A commercial CGM device (Sinocare) is used for routine glucose detection within the range of 2-25 mM, and an organic electrochemical transistor (OECT)-based sensor is developed as a backup glucose-sensing module. To prepare the PEDOT:PSS ink used as the channel material, pristine PEDOT:PSS dispersion is mixed with GOPS (1 w/w %), glycerol (5 v/v %), and DBSA (0.1 v/v %) and vortexed for 3 minutes. The resulting suspension is filtered through a 0.45 μm PTFE membrane to remove aggregates and prevent nozzle clogging during printing. The gate, source, and drain electrodes are fabricated by depositing a thin gold layer onto 3D-printed microneedles via mask-assisted e-beam evaporation. The PEDOT:PSS channel is then inkjet-printed between the source and drain electrodes and dried at 110° C. for 15 minutes, after which a UV-curable resin is applied as an insulating layer. Hollow microneedles for interstitial fluid (ISF) extraction are fabricated using a high-resolution microArch™ S240 3D printing system and UV-cured to enhance mechanical strength and durability.

To fabricate the glucose-sensing gate electrodes, 30 mg of glucose oxidase (GOx) is dissolved in 1 mL of PBS. Separately, 50 mg of chitosan and 50 μL of acetic acid are dispersed in 10 mL of deionized water and stirred at 500 rpm for 12 hours at 60° C. Then, 1 mL of the GOx solution is mixed with 1 mL of the chitosan solution and sonicated for 20 minutes before use. For ferrocene modification, 18.6 mg of ferrocene is dissolved in 10 mL of PBS, and 4 μL of this solution is deposited on the gate electrode and dried at room temperature for 1 hour. Subsequently, 4 μL of the GOx-chitosan mixture is applied to the electrode and dried at 4° C. for 3 hours, yielding the final glucose-sensing gate structure.

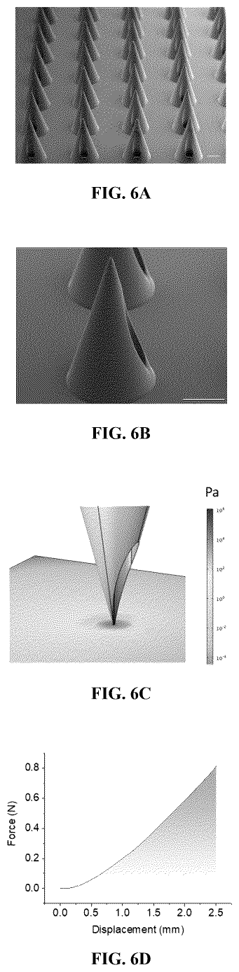

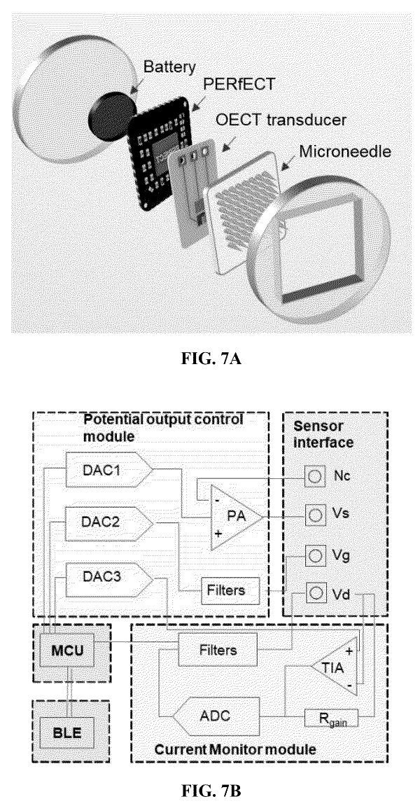

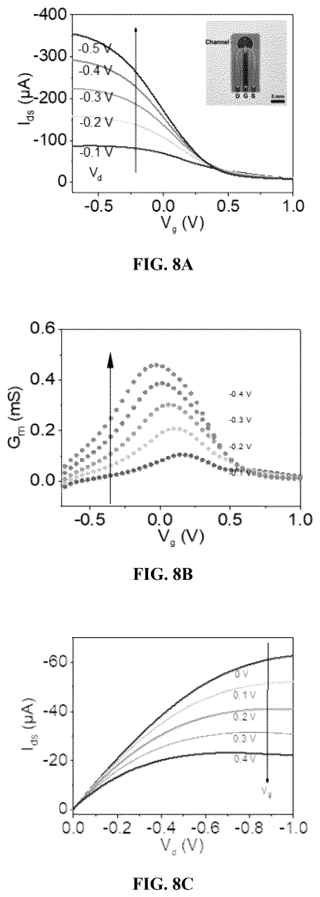

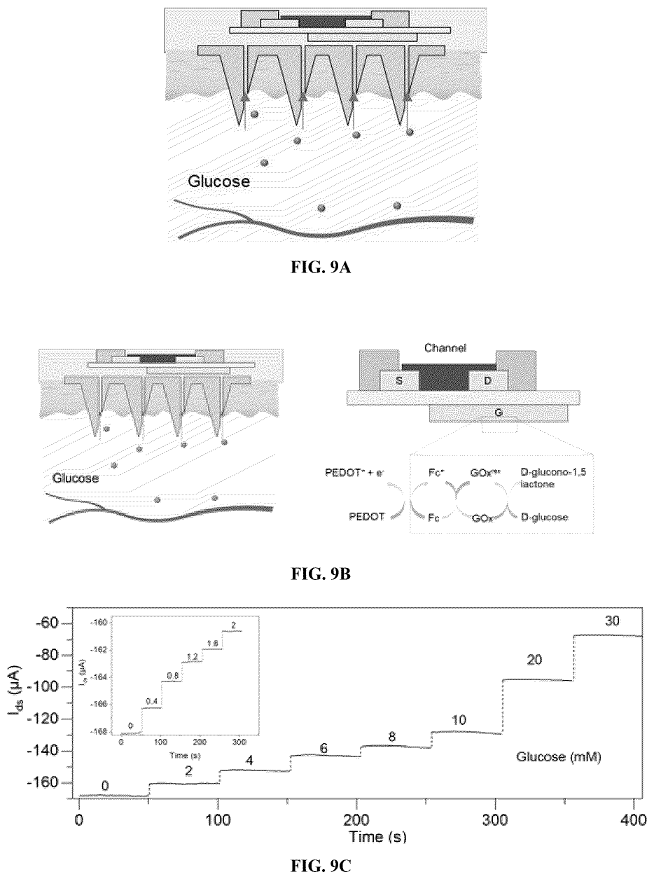

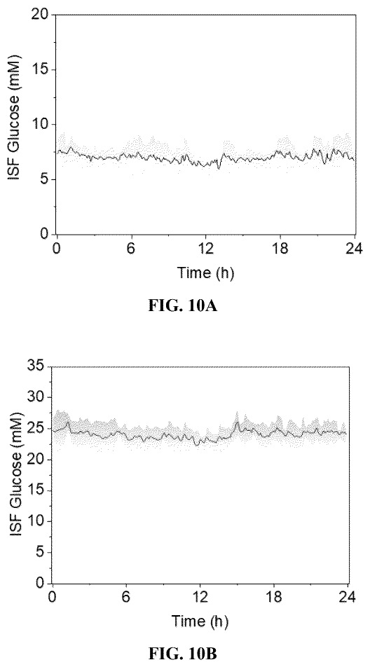

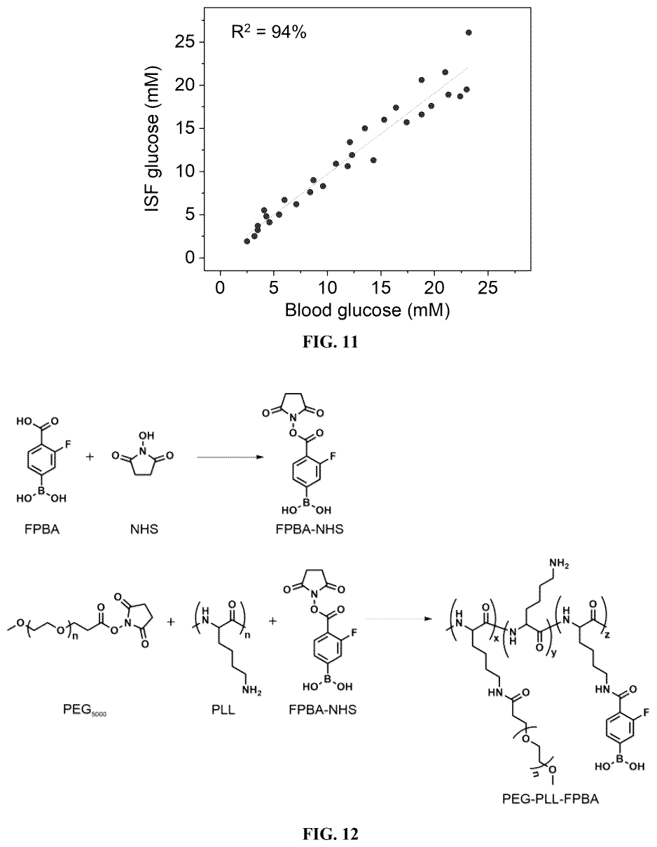

The OECT backup CGM exhibits typical depletion-mode transistor characteristics (FIGS. 6A-6D; FIGS. 7A-7B; FIGS. 8A-8C), showing a monotonic decrease in drain current (Ids) across a broad glucose concentration range from 0.2 to 40 mM (FIGS. 9A-9C). This wide detection range enables monitoring of both hyperglycemic and hypoglycemic conditions beyond the capabilities of standard commercial CGMs, thereby providing high-resolution data suitable for algorithm training. Analysis of samples collected from healthy and streptozotocin (STZ)-treated type 1 diabetic rats (FIGS. 10A-10B) across multiple glycemic states shows a strong correlation between blood glucose (BG, measured by a BG meter) and ISF glucose (ISFG, measured by the OECT sensor), with a correlation coefficient of r=0.95 (n=36) (FIG. 11). These results indicate that ISF glucose levels reliably reflect blood glucose levels, supporting their use in glucose management and closed-loop control.

### Example 3. Development of GRI

To develop the GRI, recombinant human insulin (RHI) is covalently modified with gluconic acid to produce an insulin analogue bearing a diol-containing gluconic acid moiety (FIG. 12). This gluconic-acid-modified insulin, referred to as Glu-insulin, forms a stable complex with poly-L-lysine functionalized with 4-carboxy-3-fluorobenzeneboronic acid (PLL-FPBA) through dynamic electrostatic interactions and reversible phenylboronic acid-diol complexation. For fabrication of the GRI complex, the polymer and RHI are combined at a mass ratio of 10:1. Specifically, 10 mg of polymer is dissolved in 1 mL of deionized (DI) water, while 10 mg of RHI is dissolved in 1 mL of acidic solution (prepared by mixing 60 μL of 1 M HCl with 1 mL DI water). Equal volumes of the polymer and RHI solutions are mixed, and the pH is rapidly adjusted to 7.4 using sodium hydroxide.

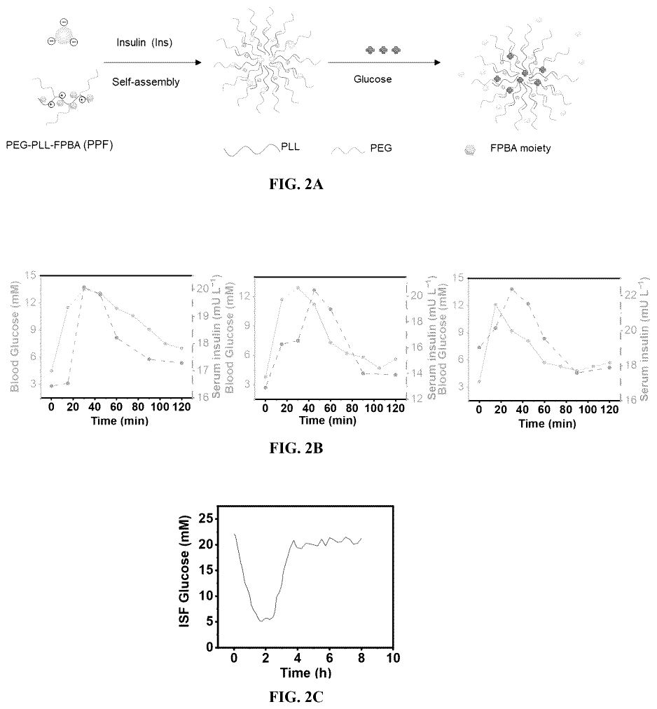

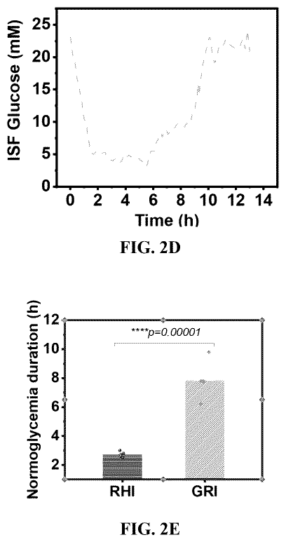

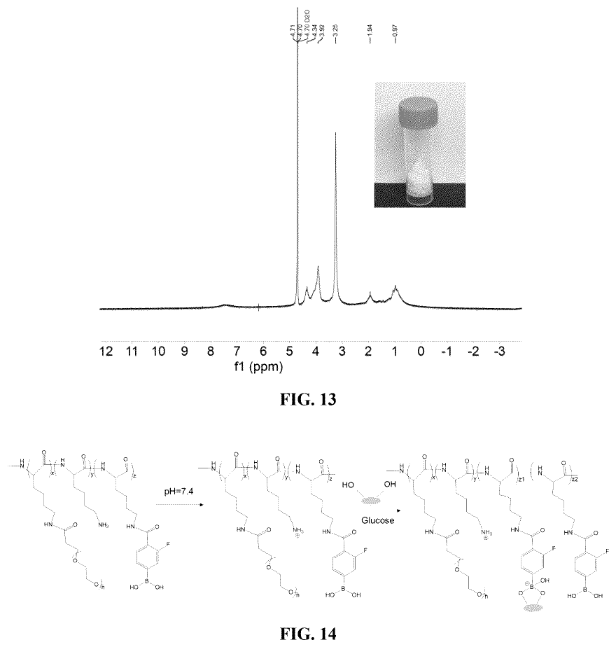

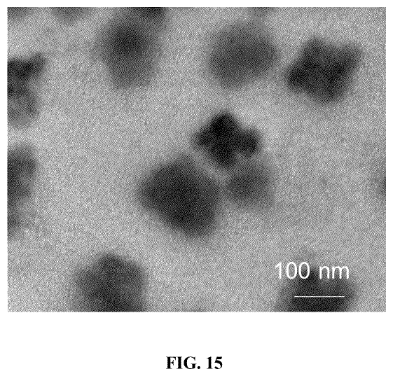

Under normoglycemic conditions, strong electrostatic interactions and PBA-diol complexation restrict insulin release to only an ultra-small fraction in its free form. Successful modification of FPBA and PEG on PLL is confirmed by 1H NMR spectroscopy (FIG. 13). Under hyperglycemic conditions, glucose competitively binds to FPBA moieties, instantly reducing the positive charge density of PLL-FPBA and disrupting phenylboronate ester bonds. This dual effect weakens both electrostatic attraction and ester crosslinking between Glu-insulin and the polymer, thereby accelerating insulin release (FIG. 2A and FIG. 14). Transmission electron microscopy (TEM) imaging shows that the resulting polymer-insulin complexes form uniformly distributed nanomicelles with diameters of approximately 100-200 nm (FIG. 15).

In vivo experiments are conducted to evaluate glucose-triggered insulin release. Male Sprague-Dawley (SD) rats (300-350 g, 6-8 weeks old) are obtained from the Guangdong Medical Laboratory Animal Center. Animals are acclimated for one week in a standardized, pathogen-free environment at the Laboratory Animal Center of Guangzhou Medical University. Type 1 diabetes is induced via a single intraperitoneal injection of streptozotocin (STZ, 110 mg/kg) after a 12-hour fast. Unless otherwise specified, diabetic rats have free access to food and water and are housed under controlled temperature (20-26° C.) and humidity (50-70%). Rats are divided into groups receiving subcutaneous injections of RHI or GRI. Blood glucose (BG) levels are measured using a commercial glucose meter (Sinocare). Isoflurane anesthesia is used to minimize discomfort, and animal usage is kept to the minimum necessary.

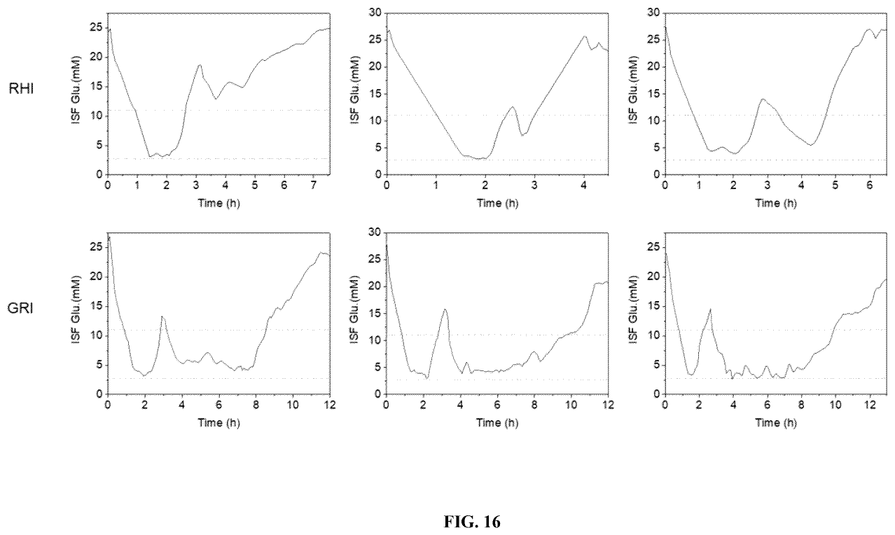

For glucose-triggered release testing, diabetic rats receive GRI via transcutaneous injection. Two hours later, glucose (1.35 g/kg) is administered (time 0 in FIG. 2B), causing BG levels to rise sharply within ~15 minutes. This hyperglycemic spike triggers a rapid increase in serum insulin (21.0±1.0 mU/L) at 30-45 minutes, which subsequently reduces BG levels. As BG levels decline to normoglycemia, negative feedback reduces insulin release from the GRI complex, with insulin levels decreasing between 45-60 minutes. To further analyze in vivo insulin dynamics, oral glucose tolerance tests are performed 2 hours after GRI or RHI administration (FIG. 16). Briefly, diabetic rats (n=3) are administered either HRI or GRI at a dose of 25 U/kg. Three hours later, all rats receive an intraperitoneal injection of glucose (0.3 g/mL in PBS, 1.5 g/kg). ISF glucose levels are continuously monitored and analyzed thereafter.

GRI-treated rats exhibit a delayed ISF glucose elevation after glucose challenge, followed by a rapid return to normoglycemia that is sustained over 5-6 hours. In contrast, RHI-treated rats show only a brief BG reduction followed by a rapid return to hyperglycemia within 1 hour.

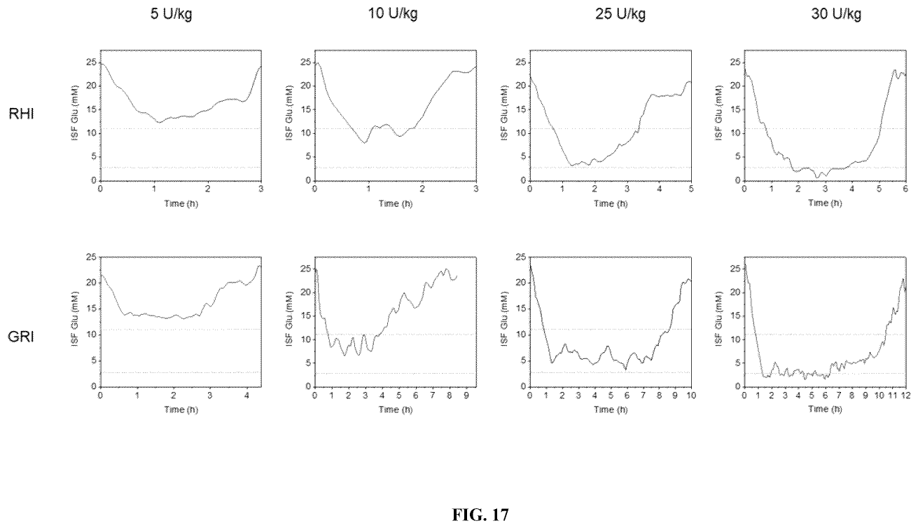

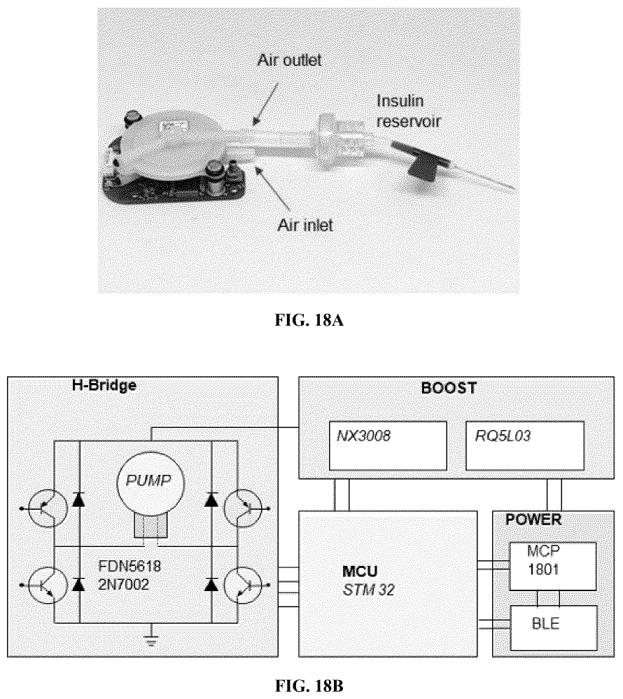

Dose-response experiments are conducted using four RHI groups and four GRI groups (FIG. 17). At equivalent insulin doses, each GRI group maintains normoglycemia for a substantially longer duration than the corresponding RHI group. Doses of 5 and 15 U/kg are insufficient to maintain normoglycemia, whereas 35 U/kg results in rebound hyperglycemia. An optimal dose of 25 U/kg achieves approximately 8 hours of normoglycemia without inducing hypoglycemia and is therefore selected as the maximum single-injection dose for subsequent studies. In five diabetic rats evaluated using 25 U/kg, both RHI and GRI injections lead to an initial drop in ISF glucose to normoglycemia within 0.5-1.5 hours. However, RHI-treated rats return to hyperglycemia within 3 hours due to limited insulin retention (FIG. 2C), whereas GRI-treated rats maintain extended normoglycemia lasting 6-10 hours (FIGS. 2D and 2E).

### Example 4. Development of the Algorithm for the Dual-Loop System

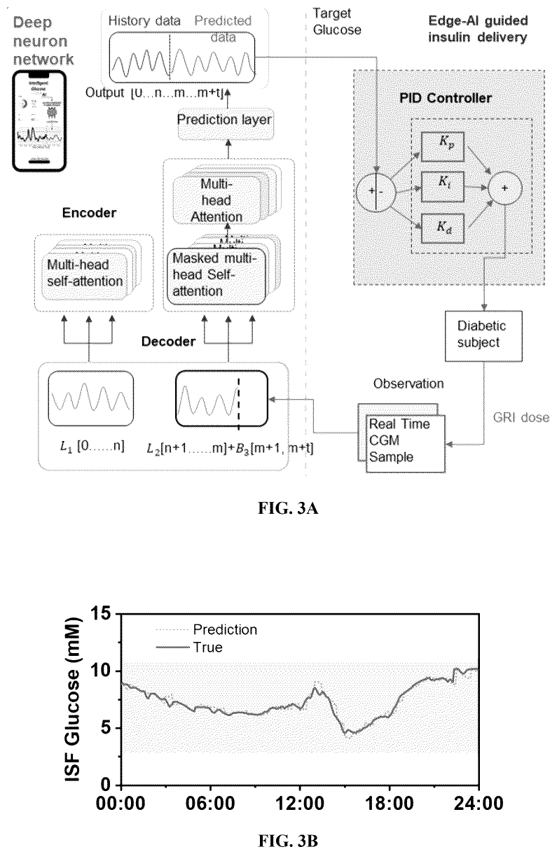

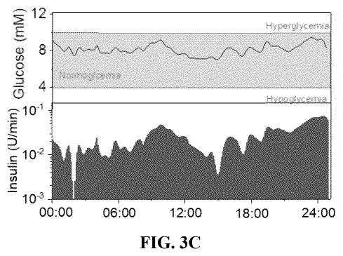

An edge-AI glucose-prediction and control algorithm is developed to integrate the CGM module and the GRI delivery system, enabling precise blood-glucose forecasting and automated GRI administration. Because traditional mathematical models are insufficient to capture the complexity of human metabolic fluctuations, a Transformer-based deep neural network (FIG. 3A, left) is implemented to construct an intelligent, fully automated closed-loop glucose-management architecture. This system supports a PID-based GRI-release mechanism (FIG. 3A, right) and achieves accurate 30-minute predictions of blood-glucose trends.

To establish this capability, a pre-trained machine-learning adaptive glucose-forecasting model is developed, employing an end-to-end Transformer architecture with an Encoder-Decoder structure. The Encoder contains two self-attention layers, while the Decoder contains a masked self-attention layer followed by an additional self-attention layer. The embedded hidden dimension is 128. Input sequences consist of 100 minutes of historical CGM data together with insulin-administration records. The Encoder processes 19 historical data inputs plus the current CGM value (L_1, 100 minutes), while the Decoder receives the most recent 10 data points (L_2, 50 minutes). The last 6 data points in the Decoder (B_3, 30 minutes) are masked to allow autoregressive prediction. The model outputs a continuous forecast of blood-glucose levels for the upcoming 30 minutes.

The predictive model is integrated with a PID closed-loop control algorithm to optimize automated GRI delivery. The PID controller includes proportional, integral, and derivative components modeled according to the following formula:

$${I}_{t} = {K}_{p} ( {G}_{t} - {G}_{B} ) + {K}_{i} \int ( {G}_{t} - {G}_{B} ) d t + {K}_{d} \frac{d G}{d t}$$

where G_{B} is the target glucose level. The proportional term Kₚ(Gt−GB) quantifies the instantaneous deviation from target glucose. The integral term Kᵢ∫(Gₜ−G_{B})dt compensates for accumulated errors and dynamically adjusts GRI output. The derivative term

$${K}_{d} \frac{d G}{d t}$$

estimates glucose-change trends, originally using dG/dt=(Gₜ−Gₜ₋₁)/5 min. When combined with the Transformer model, the derivative term is enhanced using a more accurate predictive gradient: dG/dt=(Gₜ₊₁−Gₜ₋₁)/(2×5), with Gₜ₊₁ derived from the model's forecast. This integration significantly improves anticipatory glucose-control capability.

Due to the computational efficiency of the PID module, it can be deployed directly on the insulin pump's embedded processor for real-time edge-based GRI regulation. To further enable practical deployment, Adaquant compression is applied to reduce the Transformer model to one-quarter of its original parameter size, allowing real-time operation on a smartphone. CGM prediction data are received through Bluetooth, displayed to the user, and transmitted to the insulin pump for autonomous, dual-loop edge-AI-guided GRI control.

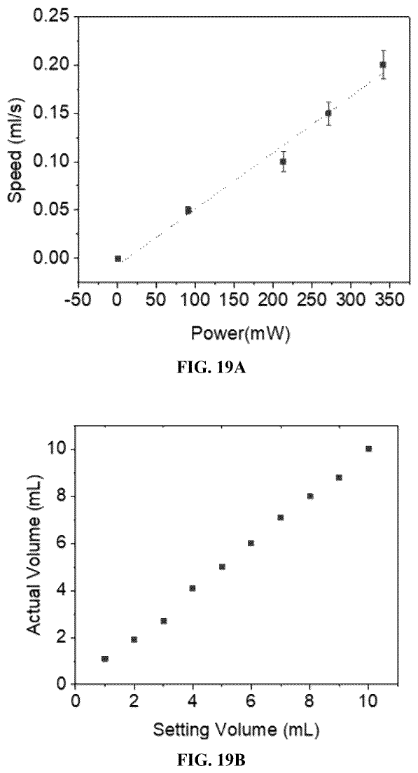

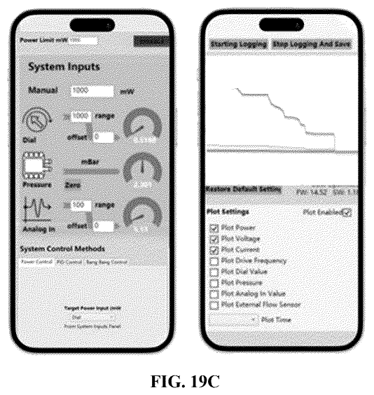

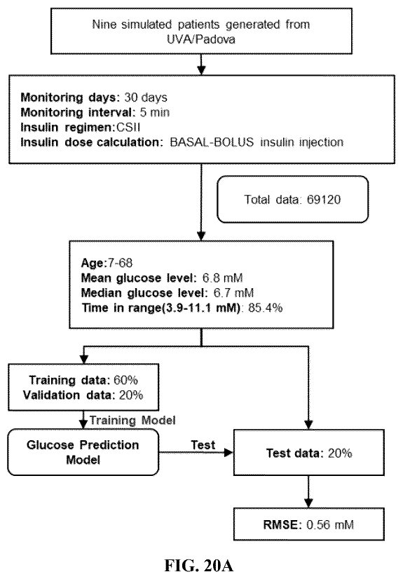

The custom insulin pump assembly consists of a syringe, a stepper motor, and an electronic printed circuit board (PCB) that together provide precise dosing capability (FIGS. 18A-18B). Pump operation is controlled wirelessly via Bluetooth (FIG. 19A), enabling adjustment of power and insulin-delivery rates. Experimental measurements of delivered solution volumes exhibit close agreement with programmed values (FIG. 19B), confirming that the pump satisfies accuracy requirements for closed-loop insulin administration.

### Example 5. Validation of the Algorithm

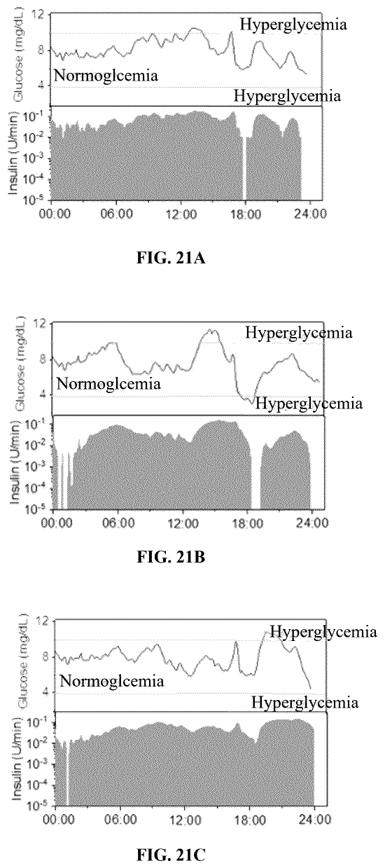

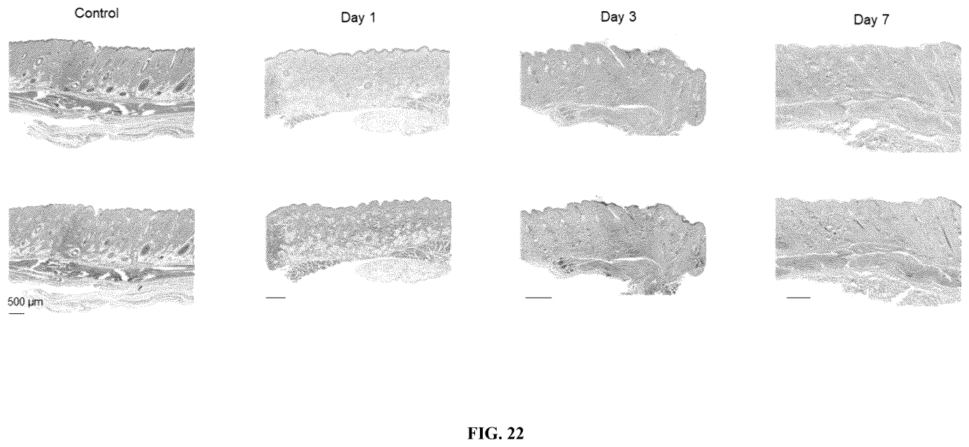

The effectiveness of the proposed AI algorithm is first evaluated within a simulation environment. Using the UVA/Padova Type 1 Diabetes simulator, eight virtual diabetic patients are generated, and simulated CGM data are continuously collected over a 30-day period. Blood-glucose variations in these virtual subjects are regulated using a conventional BASAL-BOLUS insulin-administration regimen. The dataset is then partitioned into a training set (60%), validation set (20%), and test set (20%) for model development. As shown in FIG. 3B, the model's predicted values closely track actual blood-glucose levels (BGLs) for a representative virtual patient, achieving a root-mean-square error (RMSE) of 0.5 mM on the test set. By accurately forecasting near-term glucose trajectories, the Transformer model enhances the performance of the PID controller, enabling real-time insulin-dosing adjustments based on predicted glucose dynamics (FIG. 3C). In simulation, this integrated system effectively stabilizes glucose fluctuations in all virtual patients, confirming the robustness and functional reliability of the AI-assisted dual-loop control strategy (FIGS. 21A-21C).

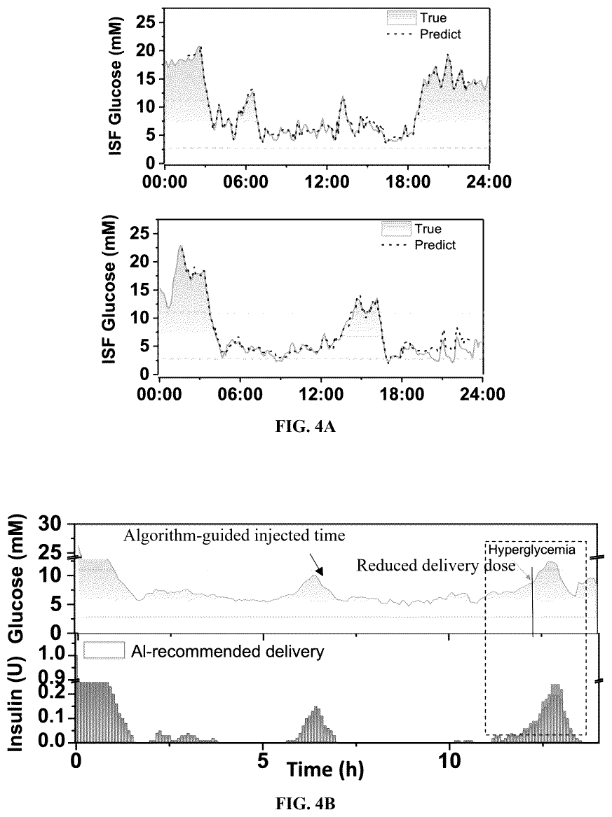

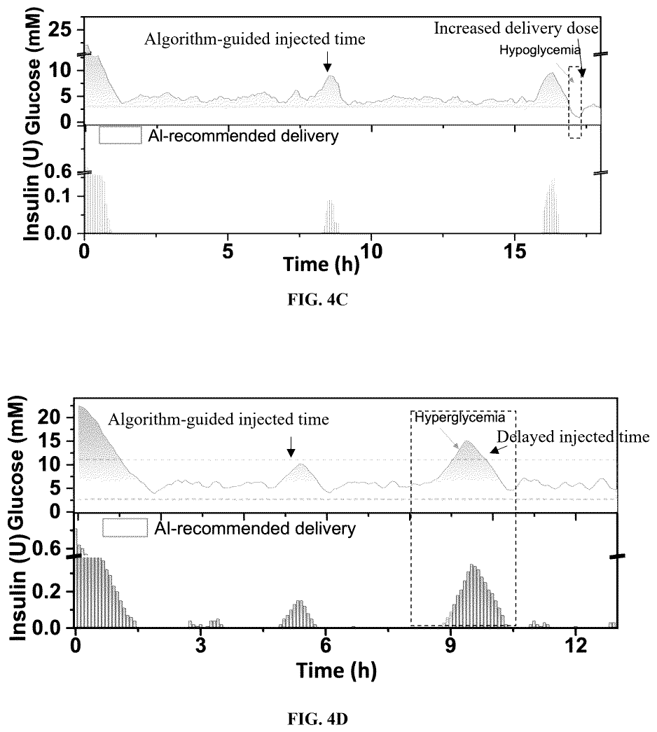

Following simulator validation, the customized algorithm is further assessed in vivo by comparing predicted ISF glucose values with experimentally measured ISF glucose in diabetic rats. The model demonstrates strong predictive accuracy, achieving an RMSE of 1.03 mM on the test dataset (FIG. 4A). To evaluate the impact of AI-guided insulin-delivery adjustments, both insulin dose and dosing window are intentionally modified according to the Edge-AI system. When insulin is delivered in accordance with Edge-AI guidance, glucose levels remain stably within the normoglycemic range (FIGS. 4B-4D, second peak). However, insufficient insulin dosing leads to hyperglycemia (FIG. 4D, third peak), whereas excessive dosing induces hypoglycemia (FIG. 4C, third peak), demonstrating the critical importance of accurate dose selection. The influence of dosing-time precision is also examined: delays in insulin administration relative to Edge-AI-recommended timing result in hyperglycemia (FIG. 4D, third peak), underscoring the necessity of timely dosing for optimal glucose control. Collectively, these results verify the robustness, precision, and practical applicability of the Edge-AI-assisted closed-loop system under real biological conditions, supporting its potential for effective and safe diabetes management.

### Example 6. In Vivo Validation

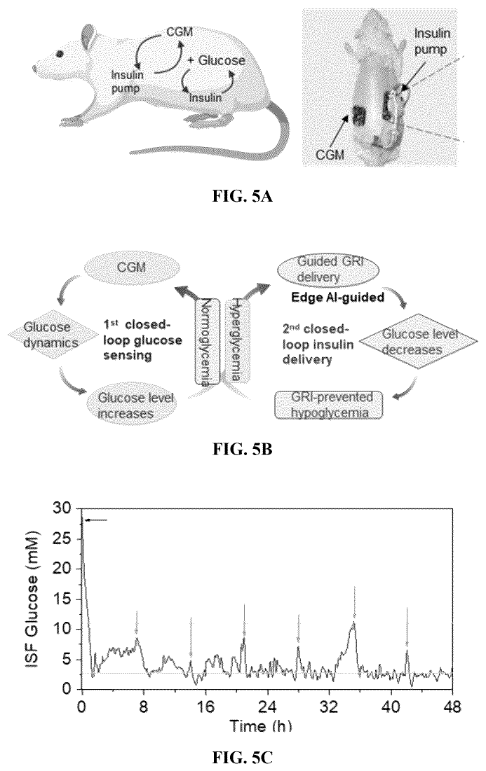

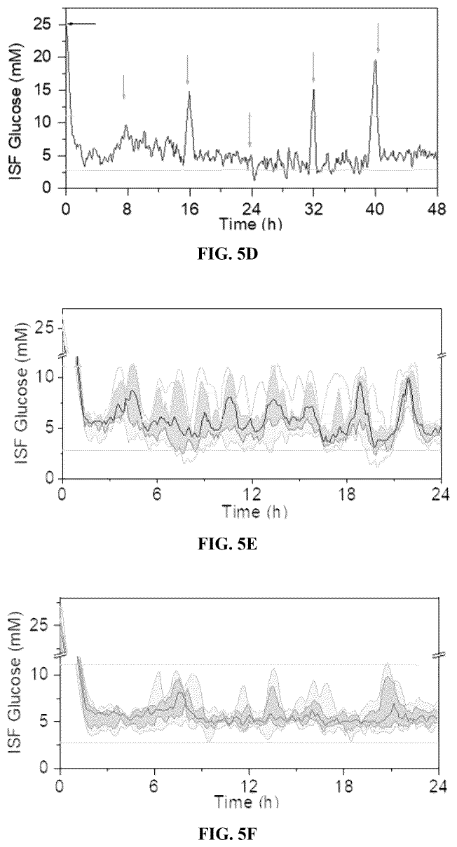

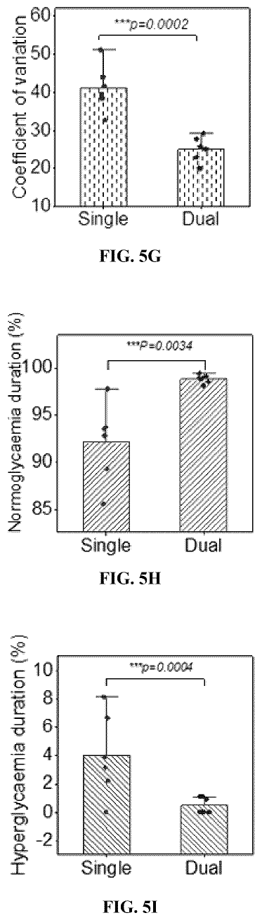

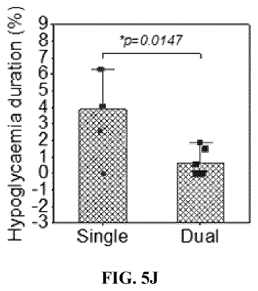

The therapeutic efficacy of the DuoLoop system is evaluated in a type 1 diabetic rat model. The integrated system—consisting of a CGM and an insulin pump—is mounted comfortably onto the rats and operates wirelessly via Bluetooth, minimizing interference with their natural behavior (FIG. 5A). The overall workflow of the DuoLoop system is shown in FIG. 5B. When the CGM detects an upward glucose trend approaching hyperglycemia, the algorithm activates the insulin pump to deliver an appropriate dose of insulin within a defined time window. During hyperglycemic episodes, insulin delivery is accelerated, and once normoglycemia is restored, the GRI autonomously reduces its release rate, thereby maintaining stable glucose levels and preventing hypoglycemia.

The DuoLoop system—which integrates both electrical and chemical closed-loop control—exhibits superior glycemic management compared to a traditional SinLoop system. As an initial comparison, GRI is administered manually at 5 U (~15 U/kg) every 7-8 hours to mimic a chemical-only closed-loop configuration (FIG. 5C and FIG. 5D). Although this approach maintains glucose levels near the normoglycemic range, episodes of hyperglycemia and hypoglycemia still occur. These results indicate that while GRI can modulate insulin release in response to glucose fluctuations, the absence of precise, AI-guided dosing results in substantial glycemic variability.

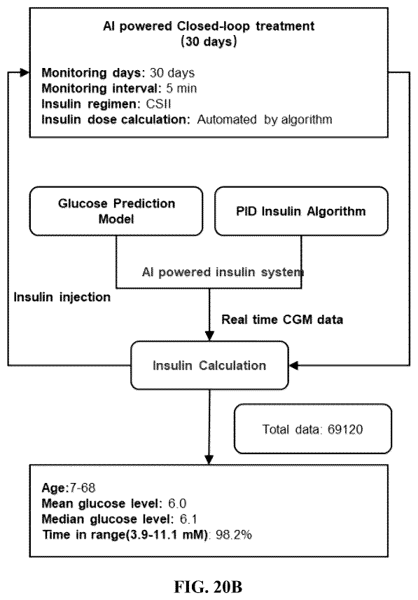

Comparative analysis of glycemic profiles obtained using the traditional electrical closed-loop system versus the DuoLoop dual-loop system (FIGS. 20A-20B, FIG. 5E, and FIG. 5F) further highlights the advantages of the invention. The SinLoop approach requires multiple injections per day, whereas the DuoLoop system requires fewer injections because GRI provides a longer duration of normoglycemia at equivalent doses (as demonstrated in FIG. 2E). Additionally, DuoLoop significantly reduces glucose fluctuations, as reflected by a lower coefficient of variation (25.14 vs. 41.22, FIG. 5G). Clinical research consistently indicates that minimizing glucose variability is essential for long-term diabetes management. Benefiting from GRI's self-regulating insulin-release mechanism, the DuoLoop system maintains a substantially higher percentage of normoglycemic periods (98.82% vs. 92.10%, calculated from 1.5-24 h), and exhibits markedly fewer episodes of hypoglycemia (0.65% vs. 3.89%) and hyperglycemia (0.52% vs. 4.01%) compared to the traditional SinLoop system (FIGS. 5H-5J).

Additionally, hematoxylin and eosin (H&E) staining and Masson's trichrome staining are performed to assess local tissue responses at injection sites. Diabetic rats receive subcutaneous injections of either PBS (control) or GRI (experimental, 25 U/kg, n=3) on days 1, 3, 5, and 7. Subcutaneous tissues from injection sites are excised, fixed in 4% paraformaldehyde, embedded in paraffin, sectioned, and stained. Stained tissue sections are imaged using a digital slide scanner and analyzed using Olympus Image Viewer software.

Histological analysis reveals no significant neutrophil infiltration, fibrosis, or abnormal collagen deposition at the injection sites, confirming the biocompatibility and safety of the DuoLoop system for in vivo application (FIG. 23).

In summary, traditional single closed-loop insulin delivery systems are susceptible to erroneous glucose readings, which may lead to insulin overdosing, hypoglycemia, and potentially severe clinical complications. To overcome these limitations, the present invention provides a DuoLoop system that integrates two complementary closed-loop mechanisms. The first closed loop uses a commercial wearable CGM to automate insulin delivery. The second closed loop relies on GRI, in which the insulin release rate self-adjusts according to real-time blood-glucose levels. An AI-based algorithm, trained on extensive glucose datasets, links these two components by accurately predicting glucose trajectories and determining optimal GRI dosing and timing. In vivo studies demonstrate that the AI-guided DuoLoop system markedly reduces glycemic fluctuations and significantly lowers the incidence of both hyperglycemia and hypoglycemia compared to traditional single-loop systems.

The functional units and modules of the systems and/or methods in accordance with the embodiments disclosed herein may be implemented using computing devices, computer processors, or electronic circuitries including but not limited to application specific integrated circuits (ASIC), field programmable gate arrays (FPGA), microcontrollers, graphical processing units (GPU), and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance to the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A wearable dual closed-loop insulin delivery system integrating a chemical closed-loop and an electronic closed-loop, comprising:
a continuous glucose monitor (CGM) configured to obtain a series of real-time blood glucose concentrations of a subject;
a glucose-responsive insulin (GRI) delivery device configured to administer insulin to the subject at a release rate modulated by the real-time blood glucose concentrations, wherein the GRI delivery device comprises a GRI formulation that autonomously modulates its insulin-release rate in response to the subject's real-time glucose concentration, thereby forming a chemical closed-loop; and
a controller comprising one or more processors and a machine-learning adaptive glucose forecasting model implemented thereon, wherein the model comprising an Encoder-Decoder architecture configured to:
receive the series of real-time glucose concentrations from the CGM;
generate a predicted glycemia dynamics of the subject for at least a subsequent time period prediction horizon; and
generate dosing-control signals based on the predicted glycemia dynamics to adjust an insulin-dosing amount and an insulin-dosing timing of the GRI delivery device, thereby forming an electronic closed-loop;
wherein the controller is operatively connected to the CGM to receive the real-time glucose concentrations and operatively connected to the GRI delivery device to transmit the dosing-control signals.

2. The wearable dual closed-loop insulin delivery system of claim 1, wherein the wearable system is compatible with a smart device comprising a smartphone, a smartwatch, and smart glasses.

3. The wearable dual closed-loop insulin delivery system of claim 1, the machine-learning adaptive glucose forecasting model is pre-trained using a dataset comprising continuous glucose monitoring data and insulin-administration records.

4. The wearable dual closed-loop insulin delivery system of claim 1, wherein the GRI delivery device reduces the occurrence of hypoglycemia by automatically decreasing insulin release in response to a glucose level declining event in the series of real-time interstitial fluid glucose concentrations.

5. The wearable dual closed-loop insulin delivery system of claim 1, wherein the Encoder comprises two self-attention layers and the Decoder comprises a masked self-attention layer followed by a self-attention layer.

6. The wearable dual closed-loop insulin delivery system of claim 3, wherein the continuous glucose monitoring data comprises at least 100 minutes of historical CGM glucose measurements together with corresponding insulin-administration records provided as input to pre-train the machine-learning adaptive glucose forecasting model.

7. The wearable dual closed-loop insulin delivery system of claim 1, wherein the machine-learning adaptive glucose forecasting model communicates the predicted glycemia dynamics to the GRI delivery device via a wireless communication interface.

8. The wearable dual closed-loop insulin delivery system of claim 1, wherein the controller further comprises a proportional-integral-derivative (PID) controller executed by the processors and configured to receive the predicted glycemia dynamics and generate the dosing-control signals.

9. The wearable dual closed-loop insulin delivery system of claim 1, wherein the CGM comprises an organic electrochemical transistor (OECT) integrated with a microneedle array for interstitial fluid extraction.

10. The wearable dual closed-loop insulin delivery system of claim 9, wherein the CGM detects glucose concentrations in a range from 0.2 mM to 40 mM.

11. The wearable dual closed-loop insulin delivery system of claim 1, wherein detection of a predicted hyperglycemia event within the predicted glycemia dynamics triggers a pre-emptive insulin-delivery action by the GRI delivery device to prevent or mitigate hyperglycemia.

12. The wearable dual closed-loop insulin delivery system of claim 1, wherein the GRI formulation comprises gluconic-acid-modified insulin complexed with poly-L-lysine functionalized with phenylboronic acid groups.

13. A method for regulating insulin delivery in a subject using the wearable dual closed-loop insulin delivery system of claim 1, the method comprising:
obtaining a series of real-time glucose measurements from the CGM;
predicting glucose levels for at least a subsequent time period using the machine-learning adaptive glucose forecasting model; and
modulating glucose-responsive insulin release from the GRI delivery device based on the predicted glucose levels so as to maintain euglycemia in the subject.

14. The method of claim 13, wherein detection of a predicted hyperglycemia event within the predicted glucose levels causes the controller to initiate a pre-emptive GRI-dosing command to the glucose-responsive insulin delivery device to prevent or reduce hyperglycemic excursions.

15. The method of claim 13, wherein detection of a predicted hypoglycemia event within the predicted glucose levels causes the controller to suppress, reduce, or temporarily suspend insulin release from the glucose-responsive insulin delivery device to avoid hypoglycemic undershoot.
